(19) United States
(12) Patent Application Publication
(10) Pub. No.: US 2025/0022582 A1
(43) Pub. Date: Jan. 16, 2025

(54) APPARATUS AND METHOD FOR DEEP LEARNING-BASED MEDICAL IMAGE STYLE NEUTRALIZATION

(71) Applicant: CLARIPI INC., Seoul (KR)

(72) Inventors: Ah Yeong LEE, Seoul (KR); Jong Hyo KIM, Seoul (KR)

(21) Appl. No.: 18/771,538

(22) Filed: Jul. 12, 2024

(30) Foreign Application Priority Data
Jul. 13, 2023 (KR) 10-2023-0091370

(51) Int. Cl. G16H 30/40; G06T 11/00; G16H 30/20

(52) U.S. Cl. CPC G16H 30/40; G06T 11/003; G16H 30/20

(57) ABSTRACT

Disclosed is a method of deep learning-based medical image style neutralization for generating a neutralized image to be input to an artificial intelligence-based diagnosis support program includes: obtaining a medical image for processing from an outside; and generating a neutralized image by inputting the medical image for the processing to a style neutralization deep learning model trained in advance to neutralize imaging characteristics of the medical image for the processing, wherein the style neutralization deep learning model includes a plurality of style neutralization deep learning models, and the style neutralization deep learning model corresponding to the imaging characteristics of the medical image for the processing performs the neutralization of the medical image for the processing.

### CROSS REFERENCE TO RELATED APPLICATION

Priority to Korean patent application number 10-2023-0091370 filed on Jul. 13, 2023 the entire disclosure of which is incorporated by reference herein, is claimed.

### BACKGROUND OF THE INVENTION

### Field of the Invention

The disclosure relates to an apparatus and method for deep learning-based medical image style neutralization, and more particularly to an apparatus and method for deep learning-based medical image style neutralization to neutralize image style characteristics.

### Description of the Related Art

In general, an X-ray, a computed tomography (CT), a magnetic resonance imaging (MRI), and the like medical apparatuses are used to acquire medical images. In modern medicine, the medical images acquired through such medical apparatuses are used as a very important basis for the presence and characteristics of lesions to make decisions in a process of diagnosing and treating a patient.

With the recent advancement of artificial intelligence technology, various technologies have been researched to assist in making decision based on artificial intelligence. For example, an artificial intelligence-based diagnosis support technology has been disclosed in Korean Patent No. 10-2108401 (titled “IDENTIFICATION SERVER BY ARTIFICIAL INTELLIGENCE AND IMAGE PROCESSING SYSTEM BASED ON PACS INCLUDING THE SAME,” and registered on Apr. 29, 2020). In this related art, medical images acquired from various medical apparatuses are input to an artificial intelligence model to identify the presence of a lesion, and commence with medical treatment quickly based on an identification result.

However, an input image input to an artificial intelligence-based diagnosis support program is varied in imaging characteristics depending on device types, imaging techniques, etc. Accordingly, when input images different in the imaging characteristics are applied to the artificial intelligence-based diagnosis support program, a problem arises in that diagnosis support performance is varied depending on the input images.

### SUMMARY OF THE INVENTION

An aspect of the disclosure is to provide an apparatus and method for deep learning-based medical image style neutralization, in which input images different in imaging characteristics are standardized by neutralizing the imaging characteristics of the input images.

According to an embodiment of the disclosure, a method of deep learning-based medical image style neutralization for generating a neutralized image to be input to an artificial intelligence-based diagnosis support program includes: obtaining a medical image for processing from an outside; and generating a neutralized image by inputting the medical image for the processing to a style neutralization deep learning model trained in advance to neutralize imaging characteristics of the medical image for the processing, wherein the style neutralization deep learning model includes a plurality of style neutralization deep learning models, and the style neutralization deep learning model corresponding to the imaging characteristics of the medical image for the processing performs the neutralization of the medical image for the processing.

Each deep learning model may include: an inverse-transformation deep learning model trained based on raw data acquired from a patient and a reconstruction image reconstructed by reflecting imaging characteristics in the raw data, and configured to output the raw data of the medical image for the processing upon receiving the medical image for the processing; and an imitation deep learning model trained based on the raw data acquired from the patient and the reconstruction image reconstructed by reflecting the imaging characteristics in the raw data, and configured to output the neutralized image for the medical image upon receiving the raw data of the medical image for the processing.

In the inverse-transformation deep learning model and the imitation deep learning model, a parameter related to the imaging characteristics of the medical image may be changed, and the parameter may include at least one of tube voltage (kVp), tube current (mAs), detection quantum efficiency (DQE), noise, focal spots, compression force, and post processing methods.

In training the inverse-transformation deep learning model, the method may further include: obtaining raw data from a patient; obtaining raw data, in which imaging characteristics are reflected, by reflecting the imaging characteristics in the raw data; obtaining a reconstruction image, in which the imaging characteristics are reflected, by reconstructing the raw data to reflect the imaging characteristics; and training the inverse-transformation deep learning model by pairing the raw data and the reconstruction image to output the raw data of the medical image for the processing upon inputting the medical image for the processing to the inverse-transformation deep learning model.

In training the imitation deep learning model, the method may further include: obtaining raw data from a patient; obtaining raw data, in which imaging characteristics are reflected, by reflecting the imaging characteristics in the raw data; obtaining a reconstruction image, in which the imaging characteristics are reflected, by reconstructing the raw data to reflect the imaging characteristics; and training the imitation deep learning model by pairing the raw data and the reconstruction image to output a neutralized image of the medical image for the processing upon inputting the raw data of the medical image for the processing to the imitation deep learning model.

Each of the inverse-transformation deep learning model and the imitation deep learning model may include a plurality of deep learning models which are different from each other in the imaging characteristics to be converted.

The inverse-transformation deep learning model may include: a first inverse-transformation deep learning model trained based on the raw data obtained from the patient and a reconstruction image reconstructed by reflecting a first imaging characteristic in the raw data; and a second inverse-transformation deep learning model trained based on the raw data obtained from the patient and a reconstruction image reconstructed by reflecting a second imaging characteristic in the raw data, and the imitation deep learning model may include: a first imitation deep learning model trained based on the raw data obtained from the patient and a reconstruction image reconstructed by reflecting a first imaging characteristic in the raw data; and a second imitation deep learning model trained based on the raw data obtained from the patient and a reconstruction image reconstructed by reflecting a second imaging characteristic in the raw data.

The style neutralization deep learning model may include: a first style neutralization deep learning model to which the first inverse-transformation deep learning model and the first imitation deep learning model are connected; and a second style neutralization deep learning model to which the second inverse-transformation deep learning model and the second imitation deep learning model are connected.

Meanwhile, according to an embodiment of the disclosure, an apparatus for deep learning-based medical image style neutralization includes a processing unit configured to generate a neutralized image to be input to an artificial intelligence-based diagnosis support program, the processing unit being configured to: obtain a medical image for processing from an outside; and generate a neutralized image by inputting the medical image for the processing to a style neutralization deep learning model trained in advance to neutralize imaging characteristics of the medical image for the processing, wherein the style neutralization deep learning model includes a plurality of style neutralization deep learning models, and the style neutralization deep learning model corresponding to the imaging characteristics of the medical image for the processing performs the neutralization of the medical image for the processing.

Each deep learning model may include: an inverse-transformation deep learning model trained based on raw data acquired from a patient and a reconstruction image reconstructed by reflecting imaging characteristics in the raw data, and configured to output the raw data of the medical image for the processing upon receiving the medical image for the processing; and an imitation deep learning model trained based on the raw data acquired from the patient and the reconstruction image reconstructed by reflecting the imaging characteristics in the raw data, and configured to output the neutralized image for the medical image upon receiving the raw data of the medical image for the processing.

In the inverse-transformation deep learning model and the imitation deep learning model, a parameter related to the imaging characteristics of the medical image may be changed, and the parameter may include at least one of tube voltage (kVp), tube current (mAs), detection quantum efficiency (DQE), noise, focal spots, compression force, and post processing methods.

In training the inverse-transformation deep learning model, raw data may be obtained from a patient; raw data, in which imaging characteristics are reflected, may be obtained by reflecting the imaging characteristics in the raw data; a reconstruction image, in which the imaging characteristics are reflected, may be obtained by reconstructing the raw data to reflect the imaging characteristics; and the inverse-transformation deep learning model may be trained by pairing the raw data and the reconstruction image to output the raw data of the medical image for the processing upon inputting the medical image for the processing to the inverse-transformation deep learning model.

In training the imitation deep learning model, raw data may be obtained from a patient; raw data, in which imaging characteristics are reflected, may be obtained by reflecting the imaging characteristics in the raw data; a reconstruction image, in which the imaging characteristics are reflected, may be obtained by reconstructing the raw data to reflect the imaging characteristics; and the imitation deep learning model may be trained by pairing the raw data and the reconstruction image to output a neutralized image of the medical image for the processing upon inputting the raw data of the medical image for the processing to the imitation deep learning model.

Each of the inverse-transformation deep learning model and the imitation deep learning model may include a plurality of deep learning models which are different from each other in the imaging characteristics to be converted.

The inverse-transformation deep learning model may include: a first inverse-transformation deep learning model trained based on the raw data obtained from the patient and a reconstruction image reconstructed by reflecting a first imaging characteristic in the raw data; and a second inverse-transformation deep learning model trained based on the raw data obtained from the patient and a reconstruction image reconstructed by reflecting a second imaging characteristic in the raw data, and the imitation deep learning model may include: a first imitation deep learning model trained based on the raw data obtained from the patient and a reconstruction image reconstructed by reflecting a first imaging characteristic in the raw data; and a second imitation deep learning model trained based on the raw data obtained from the patient and a reconstruction image reconstructed by reflecting a second imaging characteristic in the raw data.

The style neutralization deep learning model may include: a first style neutralization deep learning model to which the first inverse-transformation deep learning model and the first imitation deep learning model are connected; and a second style neutralization deep learning model to which the second inverse-transformation deep learning model and the second imitation deep learning model are connected.

According to the disclosure, an apparatus and method for deep learning-based medical image style neutralization have an effect on converting input images different in imaging characteristics into neutralized images that exhibit optimal performance for an artificial intelligence diagnosis support program.

The technical effects of the disclosure are not limited to the aforementioned effects, and other unmentioned technical effects may become apparent to those skilled in the art from the following description.

### BRIEF DESCRIPTION OF THE DRAWINGS

FIG. 1 is a conceptual view schematically showing a deep learning-based medical image style neutralization apparatus according to an embodiment,

FIG. 2 is a conceptual view showing that a neutralized image is generated using a deep learning-based medical image style neutralization apparatus according to an embodiment,

FIG. 3 is a conceptual view showing that a deep learning model applied to a deep learning-based medical image style neutralization apparatus according to an embodiment acquires training data,

FIG. 4 is a conceptual view showing that an inverse-transformation deep learning model applied to a deep learning-based medical image style neutralization apparatus according to an embodiment is trained,

FIG. 5 is a conceptual view showing that an imitation deep learning model applied to a deep learning-based medical image style neutralization apparatus according to an embodiment is trained,

FIG. 6 is a conceptual view showing an example of operating a deep learning-based medical image style neutralization apparatus according to an embodiment,

FIG. 7 is a conceptual view showing an example of optimizing the performance of a deep learning-based medical image style neutralization apparatus according to an embodiment,

FIG. 8 is a conceptual view showing performance evaluation of a deep learning-based medical image style neutralization apparatus according to an embodiment.

### DETAILED DESCRIPTION OF THE INVENTION

Hereinafter, embodiments of the disclosure will be described in detail with reference to the accompanying drawings. However, the disclosure is not limited to the embodiments set forth herein, but may be implemented in various different ways. The embodiments are provided to only complete the disclosure and allow those skilled in the art to understand the category of the disclosure. In the accompanying drawings, the shape, etc. of an element may be exaggerated for clear description, and like numerals refer to like elements.

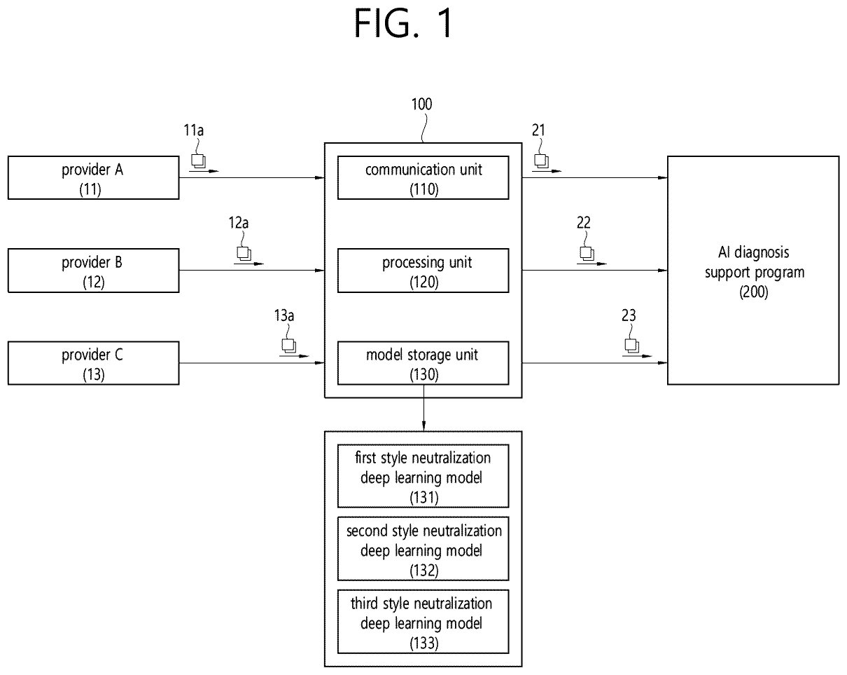

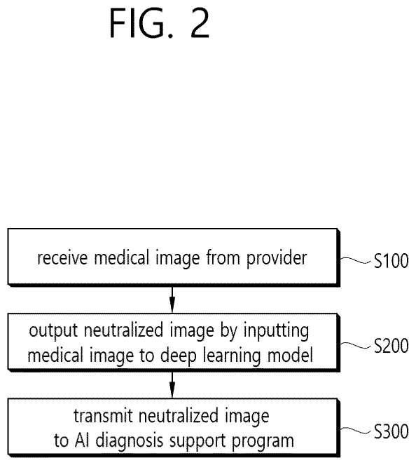

FIG. 1 is a conceptual view schematically showing a deep learning-based medical image style neutralization apparatus according to an embodiment, and FIG. 2 is a conceptual view showing that a neutralized image is generated using a deep learning-based medical image style neutralization apparatus according to an embodiment.

As shown in FIGS. 1 and 2, a deep learning-based medical image style neutralization apparatus 100 according to an embodiment (hereinafter referred to as a style neutralization apparatus) may generate neutralized images 21, 22, and 23 by standardizing medical images different in imaging characteristics.

Here, the medical images received from a plurality of providers may have different imaging characteristics due to different acquiring device types, imaging techniques, etc.

For example, a medical image A 11a received from a provider A 11 may refer to a medical image having a characteristic A, which is acquired using a device type A, an imaging technique A, etc. Further, a medical image B 12a received from a provider B 12 may refer to a medical image having a characteristic B, which is acquired using a device type B, an imaging technique B, etc. Likewise, a medical image C 13a received from a provider C 13 may refer to a medical image having a characteristic C, which is acquired using a device type C, an imaging technique C, etc.

The medical image A 11a, the medical image B 12a, and the medical image C 13a, which have different imaging characteristics, may be input to an artificial intelligence-based diagnosis support program 200, and used as basic data of reading images for a patent's lesion diagnosis. However, the medical image A 11a, the medical image B 12a, and the medical image C 13a are different in the imaging characteristics from one another, and thus exhibit different performances in the artificial intelligence-based diagnosis support program 200. Therefore, different results may be derived when the reading images respectively acquired from the medical image A 11a, the medical image B 12a, and the medical image C 13a are used as data for the lesion diagnosis.

Thus, the style neutralization apparatus 100 may generate the neutralized images 21, 22, and 23 for the medical image A 11a, the medical image B 12a, and the medical image C 13a before the medical image A 11a, the medical image B 12a, and the medical image C 13a are input to the artificial intelligence-based diagnosis support program 200, so that the neutralized images 21, 22, and 23 can be input to the artificial intelligence-based diagnosis support program 200.

For example, the style neutralization apparatus 100 may include a communication unit 110, a processing unit 120, and a model storage unit 130.

First, the communication unit 110 may receive the medical images 11a, 12a, and 13a for processing from the plurality of providers 11, 12, and 13 through wired or wireless communication (S100). In addition, the communication unit 110 may provide the neutralized images, which are generated by the style neutralization apparatus 100 based on the medical images 11a, 12a, and 13a for the processing, to the artificial intelligence-based diagnosis support program 200.

The processing unit 120 inputs the medical images 11a, 12a, and 13a for the processing, which are provided as input images from the communication unit 110, to a deep learning model trained in advance, and outputs the neutralized images from the deep learning model (S200). In this case, a plurality of deep learning models may be provided, so that the processing unit 120 can generate the neutralized image based on the deep learning model selected among the plurality of deep learning models.

The model storage unit 130 may store the plurality of deep learning models to generate the neutralized images based on the medical images 11a, 12a, and 13a for the processing. The plurality of deep learning models may include a first style neutralization deep learning model 131, a second style neutralization deep learning model 132, and a third style neutralization deep learning model 133.

For example, the first style neutralization deep learning model 131, the second style neutralization deep learning model 132, and the third style neutralization deep learning model 133 may refer to the deep learning models that receive different medical images as input data, respectively. For example, the first style neutralization deep learning model 131 may generate the neutralized image A by converting the imaging characteristics of the medical image A 11a when receiving the medical image A 11a, and the second style neutralization deep learning model 132 may generate the neutralized image B by converting the imaging characteristics of the medical image B 12a when receiving the medical image B 12a. In addition, the third style neutralization deep learning model 133 may generate the neutralized image C by converting the imaging characteristics of the medical image C 13a when receiving the medical image C 13a.

Alternatively, the first style neutralization deep learning model 131, the second style neutralization deep learning model 132 and the third style neutralization deep learning model 133 may refer to the deep learning models that output the neutralized images having different imaging characteristics, respectively.

For example, the medical image A 11a, the medical image B 12a, and the medical image C 13a may be input to the first style neutralization deep learning model 131.

Thus, the first style neutralization deep learning model 131 may convert the imaging characteristics of the medical image A 11a, the medical image B 12a, and the medical image C 13a into preset imaging characteristics. In other words, the neutralized image acquired from the medical image A 11a may be a first characteristic neutralized image A having a first imaging characteristic, and the neutralized image acquired from the medical image B 12a may be a first characteristic neutralized image B having the first imaging characteristic. In addition, the neutralized image acquired from the medical image C 13a may be a first characteristic neutralized image C having the first imaging characteristic.

In addition, the second style neutralization deep learning model 132 may convert the imaging characteristics of the medical image A 11a, the medical image B 12a, and the medical image C 13a into preset imaging characteristics. In other words, the neutralized image acquired from the medical image A 11a may be a second characteristic neutralized image A having a second imaging characteristic, and the neutralized image acquired from the medical image B 12a may be a second characteristic neutralized image B having the second imaging characteristic. In addition, the neutralized image acquired from the medical image C 13a may be a second characteristic neutralized image C having the second imaging characteristic.

Likewise, the third style neutralization deep learning model 133 may convert the imaging characteristics of the medical image A 11a, the medical image B 12a, and the medical image C 13a into preset imaging characteristics. In other words, the neutralized image acquired from the medical image A 11a may be a third characteristic neutralized image A having a third imaging characteristic, and the neutralized image acquired from the medical image B 12a may be a third characteristic neutralized image B having the third imaging characteristic. In addition, the neutralized image acquired from the medical image C 13a may be a third characteristic neutralized image C having the third imaging characteristics.

Here, a user may set up target settings in training the deep learning models to determine the imaging characteristics, the first imaging characteristic, the second imaging characteristic, and the third imaging characteristic. The style neutralization apparatus 100 transmits the generated neutralized image to the artificial intelligence-based diagnosis support program 200, thereby performing a diagnosis support (S300).

Meanwhile, a method of training the deep learning models used in the style neutralization apparatus 100 will be described below.

### Training Data Set

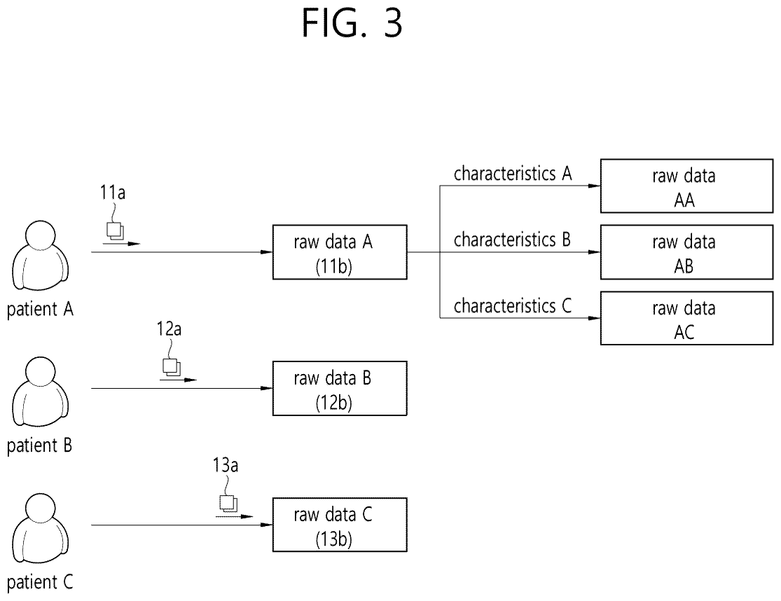

FIG. 3 is a conceptual view showing that a deep learning model applied to a deep learning-based medical image style neutralization apparatus according to an embodiment acquires training data.

As shown in FIG. 3, the acquisition of the training data for the deep learning model according to an embodiment includes a process of acquiring raw data having different characteristics from one patient, and the training data may be acquired by applying this process to a plurality of patients. Here, the raw data may include projection data which is acquired by projecting an X-ray to a patient, or a sinogram which is a set of projection data.

In more detail, the process of acquiring the training data is as follows. Raw data A 11b for a patient A may be obtained by capturing an image of the patient A, and raw data B 12b for a patent B may be obtained by capturing an image of the patient B. In addition, raw data C 13b for a patient C may be obtained by capturing an image of the patient C.

Then, the training data having different imaging characteristics may be generated from the raw data 11b, 12b, and 13b.

For example, the characteristic A related to the provider A 11, the characteristic B related to the provider B 12, and the characteristic C related to the provider C 13 may be applied to the raw data A 11b acquired from the patient A, thereby generating raw data AA related to the provider A 11, raw data AB related to the provider B 12, and raw data AC related to the provider C 13, respectively. Further, the characteristic A related to the provider A 11, the characteristic B related to the provider B 12, and the characteristic C related to the provider C 13 may be applied to the raw data B 12b acquired from the patient B, thereby generating the raw data BA related to the provider A 11, the raw data BB related to the provider B 12, and the raw data BC related to the provider C 13, respectively. Likewise, the characteristic A related to the provider A 11, the characteristic B related to the provider B 12, and the characteristic C related to the provider C 13 may be applied to the raw data C acquired from the patient C, thereby generating the raw data CA related to the provider A 11, the raw data CB related to the provider B 12, and the raw data CC related to the provider C 13.

Thus, the raw data 11b, 12b, and 13b reflecting the different imaging characteristics is obtained from each patient in acquiring the training data, thereby generating the training data set. In addition, the training data set may be used in training an inverse-transformation deep learning model and an imitation deep learning model.

### Inverse-Transformation Deep Learning Model Training

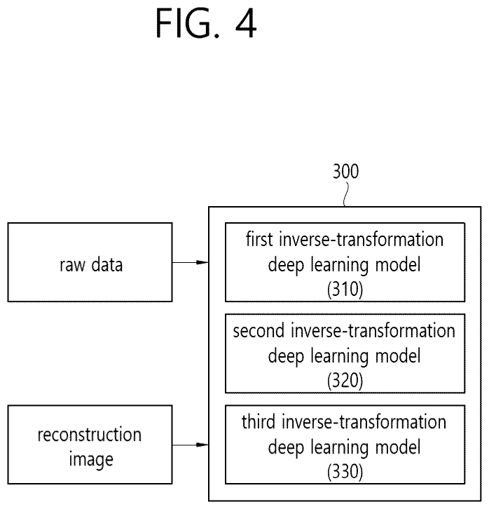

FIG. 4 is a conceptual view showing that an inverse-transformation deep learning model applied to a deep learning-based medical image style neutralization apparatus according to an embodiment is trained.

As shown in FIG. 4, the style neutralization apparatus 100 according to an embodiment includes an inverse-transformation deep learning model 300. The inverse-transformation deep learning model 300 may be trained to output a raw data image when receiving a medical image having a specific imaging characteristic. To this end, the inverse-transformation deep learning model 300 may be trained with a pair of the raw data image and a reconstruction image reconstructed from the raw data. Thus, when the reconstruction image is input to the inverse-transformation deep learning model 300, the inverse-transformation deep learning model 300 may generate the raw data image by inversely transforming the reconstruction image.

Meanwhile, the inverse-transformation deep learning model 300 may include a first inverse-transformation deep learning model 310, a second inverse-transformation deep learning model 320, and a third inverse-transformation deep learning model 330.

For example, the first inverse-transformation deep learning model 310 may be trained by training the deep learning model suitable for the imaging characteristics of the provider A 11. In this case, the raw data AA, the raw data BA, and the raw data CA acquired from the patient A, the patient B, and the patient C may be respectively paired with a reconstruction image AA, a reconstruction image BA and a reconstruction image CA reconstructed therefrom, thereby commencing with training the first inverse-transformation deep learning model 310.

For example, the second inverse-transformation deep learning model 320 may be trained by training the deep learning model suitable for the imaging characteristics of the provider B 12. In this case, the raw data AB, the raw data BB, and the raw data CB acquired from the patient A, the patient B, and the patient C may be respectively paired with the reconstruction image AB, the reconstruction image BB, and the reconstruction image CB reconstructed therefrom, thereby commencing with training the second inverse-transformation deep learning model 320.

For example, the third inverse-transformation deep learning model 330 may be trained by training the deep learning model suitable for the imaging characteristics of the provider C 13. In this case, the raw data AC, the raw data BC, and the raw data CC acquired from the patient A, the patient B, and the patient C may be respectively paired with the reconstruction image AC, the reconstruction image BC, and the reconstruction image CC reconstructed therefrom, thereby commencing with training the third inverse-transformation deep learning model 330.

Thus, the plurality of inverse-transformation deep learning models 300 may receive images respectively having specific image style characteristics and output the raw data images for the received images.

### Imitation Deep Learning Model Training

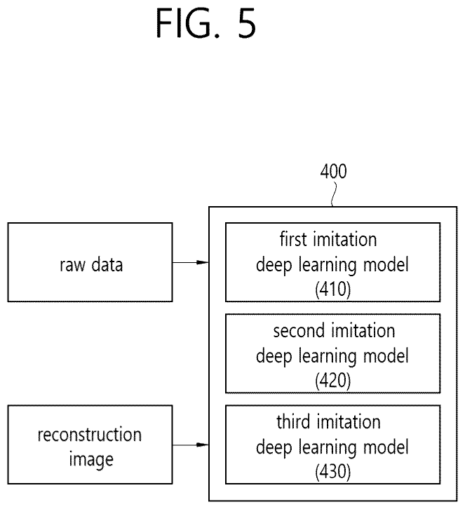

FIG. 5 is a conceptual view showing that an imitation deep learning model applied to a deep learning-based medical image style neutralization apparatus according to an embodiment is trained.

As shown in FIG. 5, the style neutralization apparatus 100 according to an embodiment includes an imitation deep learning model 400. The imitation deep learning model 400 may be trained to output a medical image having a specific imaging characteristic when receiving a raw data image. To this end, the imitation deep learning model 400 may be trained with a pair of the image and a reconstruction image reconstructed from the raw data. Thus, when the raw data image is input to the imitation deep learning model 400, the imitation deep learning model 400 may output a medical image having a specific imaging characteristic from the raw data image.

Meanwhile, the imitation deep learning model 400 may include a first imitation deep learning model 410, a second imitation deep learning model 420, and a third imitation deep learning model 430.

For example, the first imitation deep learning model 410 may trained by training the deep learning model suitable for the imaging characteristics of the provider A 11. In this case, the raw data AA, the raw data BA, and the raw data CA acquired from the patient A, the patient B, and the patient C may be paired with the reconstruction image AA, the reconstruction image BA, and the reconstruction image CA reconstructed therefrom, thereby commencing with training the first imitation deep learning model 410.

For example, the second imitation deep learning model 420 may be trained by training the deep learning model suitable for the imaging characteristics of the provider B 12. In this case, the raw data AB, the raw data BB, and the raw data CB acquired from the patient A, the patient B, and the patient C may be paired with the reconstruction image AB, the reconstruction image BB, and the reconstruction image CB reconstructed therefrom, thereby commending with training the second imitation deep learning model 420.

For example, the third imitation deep learning model 430 may be trained by training the deep learning model suitable for the imaging characteristics of the provider C 13. In this case, the raw data AC, the raw data BC, and the raw data CC acquired from the patient A, the patient B, and the patient C may be paired with the reconstruction image AC, the reconstruction image BC, and the reconstruction image CC reconstructed therefore, thereby commending with training the third imitation deep learning model 430.

Thus, the plurality of imitation deep learning models 400 may receive the raw data image and output images respectively having specific image style characteristics.

Referring back to FIGS. 1 and 2, as described above, the style neutralization apparatus 100 according to an embodiment may operate based on the first style neutralization deep learning model 131, the second style neutralization deep learning model 132, and the third style neutralization deep learning model 133.

Here, the first style neutralization deep learning model 131, the second style neutralization deep learning model 132, and the third style neutralization deep learning model 133 may be formed by a combination of the inverse-transformation deep learning model 300 and the imitation deep learning model 400.

For example, the first style neutralization deep learning model 131 may be formed by the first inverse-transformation deep learning model 310 and the first imitation deep learning model 410, and the second style neutralization deep learning model 132 may be formed by a combination of the second inverse-transformation deep learning model 320 and the second imitation deep learning model 420. In addition, the third style neutralization deep learning model 133 may be formed by a combination of the third inverse-transformation deep learning model 330 and the third imitation deep learning model 430.

Meanwhile, according to an embodiment, the neutralized image A 21, the neutralized image B 22, and the neutralized image C 23 which have the neutralized imaging characteristics are generated from the medical image A 11a, the medical image B 12a, and the medical image C 13a which are different in characteristics. In this case, various parameters of the neutralized images 21, 22, and 23 are required to be neutralized. For example, the parameters may include tube voltage (kVp), tube current (mAs), detection quantum efficiency (DQE), noise, focal spots, compression force, and post processing methods, etc. the neutralization of the parameters may cause the neutralized images 21, 22, and 23 to implement the optimal performance in the artificial intelligence-based diagnosis support program 200.

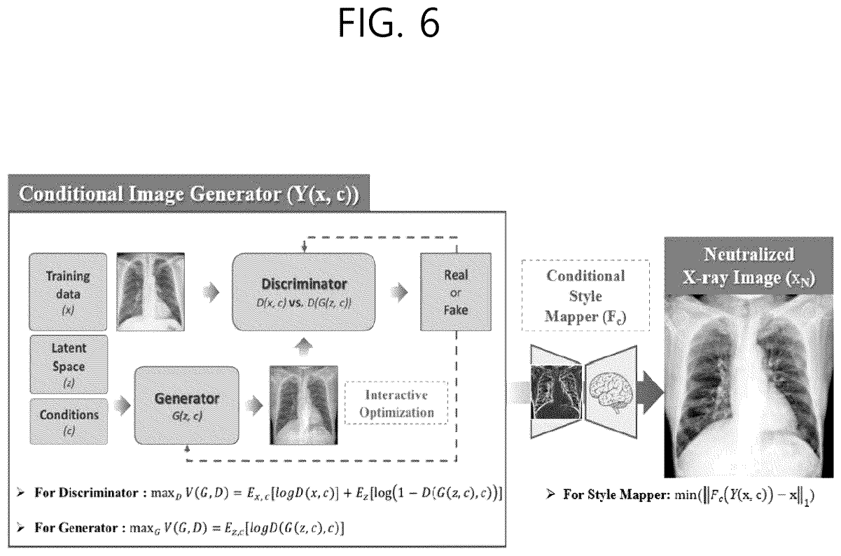

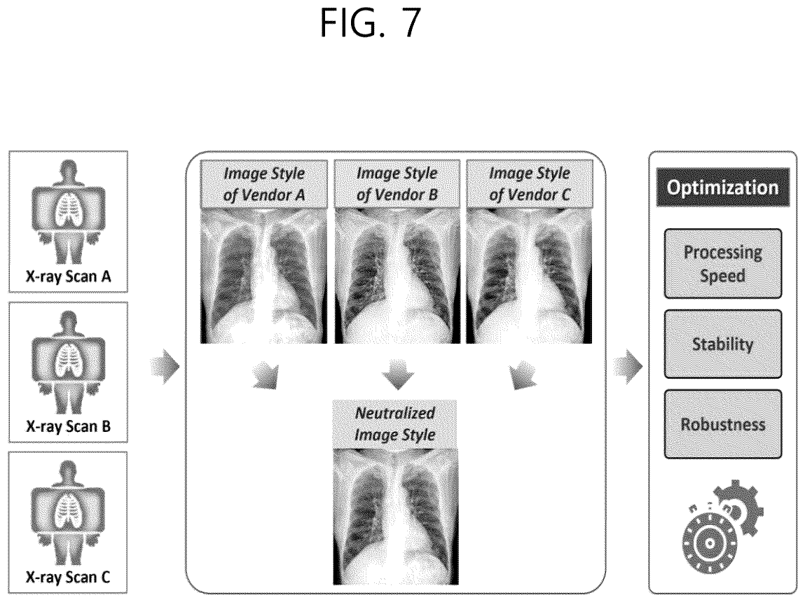

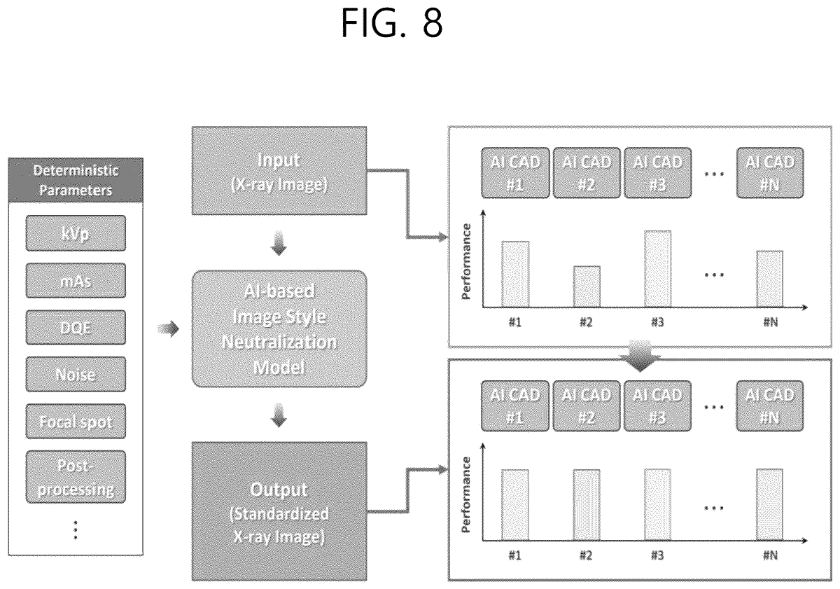

FIG. 6 is a conceptual view showing an example of operating a deep learning-based medical image style neutralization apparatus according to an embodiment, and FIG. 7 is a conceptual view showing an example of optimizing the performance of a deep learning-based medical image style neutralization apparatus according to an embodiment. In addition, FIG. 8 is a conceptual view showing performance evaluation of a deep learning-based medical image style neutralization apparatus according to an embodiment.

As shown in FIGS. 6 to 8, in the style neutralization apparatus 100 according to an embodiment, the medical image A 11a, the medical image B 12a, and the medical image C 13a different in the imaging characteristics from one another are neutralized and input to the artificial intelligence-based diagnosis support program 200.

Thus, the optimal diagnosis support process may be performed in the artificial intelligence-based diagnosis support program 200. In other words, when the neutralized image is input to the artificial intelligence-based diagnosis support program 200, the artificial intelligence-based diagnosis support program 200 may be optimized for processing speed, stability, and robustness.

Further, as shown in FIG. 8, when the medical image A 11a, the medical image B 12a, and the medical image C 13a are input to the artificial intelligence-based diagnosis support program 200, their performances are different. On the other hand, in the case of the neutralized image A 21, the neutralized image B 22, and the neutralized image C 23 converted from the medical image A 11a, the medical image B 12a and the medical image C 13a through the style neutralization apparatus 100, their performances are the same.

In this way, according to the disclosure, the apparatus and method for the deep learning-based medical image style neutralization have an effect on converting the input images different in the imaging characteristics into the neutralized images that exhibit the optimal performance for the artificial intelligence diagnosis support program.

Although a few embodiments of the disclosure have been described above and illustrated in the accompanying drawings, the embodiments should not be construed as limiting the technical spirit of the disclosure. The scope of the disclosure is limited only by the subject matters disclosed in the appended claims, and the technical spirit of the disclosure may be modified and changed in various forms by a person having ordinary knowledge in the art. Therefore, such modification and change obvious to those skilled in the art will fall within the scope of the disclosure.

What is claimed is:

1. A method of deep learning-based medical image style neutralization for generating a neutralized image to be input to an artificial intelligence-based diagnosis support program, the method comprising:
obtaining a medical image for processing from an outside; and
generating a neutralized image by inputting the medical image for the processing to a style neutralization deep learning model trained in advance to neutralize imaging characteristics of the medical image for the processing,
wherein the style neutralization deep learning model comprises a plurality of style neutralization deep learning models, and
the style neutralization deep learning model corresponding to the imaging characteristics of the medical image for the processing performs the neutralization of the medical image for the processing.

2. The method of claim 1, wherein each deep learning model comprises:
an inverse-transformation deep learning model trained based on raw data acquired from a patient and a reconstruction image reconstructed by reflecting imaging characteristics in the raw data, and configured to output the raw data of the medical image for the processing upon receiving the medical image for the processing; and
an imitation deep learning model trained based on the raw data acquired from the patient and the reconstruction image reconstructed by reflecting the imaging characteristics in the raw data, and configured to output the neutralized image for the medical image upon receiving the raw data of the medical image for the processing.

3. The method of claim 2, wherein, in the inverse-transformation deep learning model and the imitation deep learning model,
a parameter related to the imaging characteristics of the medical image is changed, and
the parameter comprises at least one of tube voltage (kVp), tube current (mAs), detection quantum efficiency (DQE), noise, focal spots, compression force, and post processing methods.

4. The method of claim 2, further comprising: in training the inverse-transformation deep learning model,
obtaining raw data from a patient;
obtaining raw data, in which imaging characteristics are reflected, by reflecting the imaging characteristics in the raw data;
obtaining a reconstruction image, in which the imaging characteristics are reflected, by reconstructing the raw data to reflect the imaging characteristics; and
training the inverse-transformation deep learning model by pairing the raw data and the reconstruction image to output the raw data of the medical image for the processing upon inputting the medical image for the processing to the inverse-transformation deep learning model.

5. The method of claim 2, further comprising: in training the imitation deep learning model,
obtaining raw data from a patient;
obtaining raw data, in which imaging characteristics are reflected, by reflecting the imaging characteristics in the raw data;
obtaining a reconstruction image, in which the imaging characteristics are reflected, by reconstructing the raw data to reflect the imaging characteristics; and
training the imitation deep learning model by pairing the raw data and the reconstruction image to output a neutralized image of the medical image for the processing upon inputting the raw data of the medical image for the processing to the imitation deep learning model.

6. The method of claim 2, wherein each of the inverse-transformation deep learning model and the imitation deep learning model comprises a plurality of deep learning models which are different from each other in the imaging characteristics to be converted.

7. The method of claim 6, wherein
the inverse-transformation deep learning model comprises:
a first inverse-transformation deep learning model trained based on the raw data obtained from the patient and a reconstruction image reconstructed by reflecting a first imaging characteristic in the raw data; and
a second inverse-transformation deep learning model trained based on the raw data obtained from the patient and a reconstruction image reconstructed by reflecting a second imaging characteristic in the raw data, and
the imitation deep learning model comprises:
a first imitation deep learning model trained based on the raw data obtained from the patient and a reconstruction image reconstructed by reflecting a first imaging characteristic in the raw data; and
a second imitation deep learning model trained based on the raw data obtained from the patient and a reconstruction image reconstructed by reflecting a second imaging characteristic in the raw data.

8. The method of claim 7, wherein the style neutralization deep learning model comprises:
a first style neutralization deep learning model to which the first inverse-transformation deep learning model and the first imitation deep learning model are connected; and
a second style neutralization deep learning model to which the second inverse-transformation deep learning model and the second imitation deep learning model are connected.

9. An apparatus for deep learning-based medical image style neutralization, the apparatus comprising a processing unit configured to generate a neutralized image to be input to an artificial intelligence-based diagnosis support program,
the processing unit being configured to:
obtain a medical image for processing from an outside; and
generate a neutralized image by inputting the medical image for the processing to a style neutralization deep learning model trained in advance to neutralize imaging characteristics of the medical image for the processing,
wherein the style neutralization deep learning model comprises a plurality of style neutralization deep learning models, and
the style neutralization deep learning model corresponding to the imaging characteristics of the medical image for the processing performs the neutralization of the medical image for the processing.

10. The apparatus of claim 9, wherein each deep learning model comprises:
an inverse-transformation deep learning model trained based on raw data acquired from a patient and a reconstruction image reconstructed by reflecting imaging characteristics in the raw data, and configured to output the raw data of the medical image for the processing upon receiving the medical image for the processing; and
an imitation deep learning model trained based on the raw data acquired from the patient and the reconstruction image reconstructed by reflecting the imaging characteristics in the raw data, and configured to output the neutralized image for the medical image upon receiving the raw data of the medical image for the processing.

11. The apparatus of claim 10, wherein, in the inverse-transformation deep learning model and the imitation deep learning model,
a parameter related to the imaging characteristics of the medical image is changed, and
the parameter comprises at least one of tube voltage (kVp), tube current (mAs), detection quantum efficiency (DQE), noise, focal spots, compression force, and post processing methods.

12. The apparatus of claim 10, wherein, in training the inverse-transformation deep learning model,
raw data is obtained from a patient;
raw data, in which imaging characteristics are reflected, is obtained by reflecting the imaging characteristics in the raw data;
a reconstruction image, in which the imaging characteristics are reflected, is obtained by reconstructing the raw data to reflect the imaging characteristics; and
the inverse-transformation deep learning model is trained by pairing the raw data and the reconstruction image to output the raw data of the medical image for the processing upon inputting the medical image for the processing to the inverse-transformation deep learning model.

13. The apparatus of claim 10, wherein, in training the imitation deep learning model,
raw data is obtained from a patient;
raw data, in which imaging characteristics are reflected, is obtained by reflecting the imaging characteristics in the raw data;
a reconstruction image, in which the imaging characteristics are reflected, is obtained by reconstructing the raw data to reflect the imaging characteristics; and
the imitation deep learning model is trained by pairing the raw data and the reconstruction image to output a neutralized image of the medical image for the processing upon inputting the raw data of the medical image for the processing to the imitation deep learning model.

14. The apparatus of claim 10, wherein each of the inverse-transformation deep learning model and the imitation deep learning model comprises a plurality of deep learning models which are different from each other in the imaging characteristics to be converted.

15. The apparatus of claim 14, wherein
the inverse-transformation deep learning model comprises:
a first inverse-transformation deep learning model trained based on the raw data obtained from the patient and a reconstruction image reconstructed by reflecting a first imaging characteristic in the raw data; and
a second inverse-transformation deep learning model trained based on the raw data obtained from the patient and a reconstruction image reconstructed by reflecting a second imaging characteristic in the raw data, and
the imitation deep learning model comprises:
a first imitation deep learning model trained based on the raw data obtained from the patient and a reconstruction image reconstructed by reflecting a first imaging characteristic in the raw data; and
a second imitation deep learning model trained based on the raw data obtained from the patient and a reconstruction image reconstructed by reflecting a second imaging characteristic in the raw data.

16. The apparatus of claim 15, wherein the style neutralization deep learning model comprises:
a first style neutralization deep learning model to which the first inverse-transformation deep learning model and the first imitation deep learning model are connected; and
a second style neutralization deep learning model to which the second inverse-transformation deep learning model and the second imitation deep learning model are connected.
